(19) United States
(12) Patent Application Publication
(10) Pub. No.: US 2023/0310391 A1
(43) Pub. Date: Oct. 5, 2023

(54) COMBINATION OF AN ANTIMUSCARINIC OR AN ANTICHOLINERGIC AGENT AND LIPOIC ACID AND USES THEREOF

(71) Applicant: Cellix Bio Private Limited, Hyderabad (IN)

(72) Inventor: Mahesh Kandula, East Godavari District, Andhra Pradesh (IN)

(73) Assignee: Cellix Bio Private Limited, Hyderabad (IN)

(21) Appl. No.: 17/993,868

(22) Filed: Nov. 23, 2022

Related U.S. Application Data
(63) Continuation of application No. 16/985,187, filed on Aug. 4, 2020, now abandoned.
Application No. 16/985,187 is a continuation of application No. PCT/IB2019/050901, filed on Feb. 5, 2019.

(30) Foreign Application Priority Data
Feb. 5, 2018 (IN) 201841004306
Mar. 5, 2018 (IN) 201841008091

(51) Int. Cl. A61K 31/439; A61K 31/223; A61K 31/385; A61K 31/4178; A61K 9/00

(52) U.S. Cl. CPC A61K 31/439; A61K 31/223; A61K 31/385; A61K 31/4178; A61K 9/0048; A61K 9/0053

(57) ABSTRACT

The present disclosure relates to pharmaceutical compositions comprising a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer thereof. The antimuscarinic or anticholinergic agent is a compound of Formula I, Formula II, or Formula III and lipoic acid is a compound of Formula IV or Formula V. The Pharmaceutical composition is a physical mixture of an antimuscarinic or an anticholinergic agent and lipoic acid.

### PRIORITY

The present application claims the benefit of the Indian provisional Application No. 201841004306 filed on Feb. 5, 2018 and Indian provisional Application No. 201841008091 filed on Mar. 5, 2018, the entire disclosures of which are relied on for all purposes and are incorporated into this application by reference.

### TECHNICAL FIELD

The present invention relates to pharmaceutical compositions and methods of using the same for the treatment of or alleviation of burning or xerostomia, particularly of the oral cavity, burning mouth syndrome and eye diseases.

### BACKGROUND OF THE INVENTION

Xerostomia, also known as dry mouth, is a condition in which an excessive dryness within the oral cavity takes place due to insufficient salivary production. Xerostomia is not a disease itself but is a side effect of a radiation to the head and neck, or a side effect of a wide variety of medications. Few common problems associated with xerostomia include but are not limited to constant sore throat, burning sensations, difficulty in speaking, swallowing and dry nasal passages, all related to the decreased level of fluids in the oral cavity.

Systemic pharmacological treatments include parasympathomimetic agents such as pilocarpine, cevimeline and bethanechol that act on β-adrenergic receptors and stimulate secretion from salivary glands. In clinical practice, they are used to treat xerostomia after radiotherapy for head and neck cancer but are associated with side effects such as headache and sweating.

Xerostomia remains an unresolved common complaint especially among the geriatric population despite seeking medical or dental consultation. Managing acute pathology often relies on the addressing underlying pathology and symptoms of the disease. There is currently a need in the art for new compositions for treating or delaying the onset of xerostomia and its associated complications progression.

Intl. Appl. No. PCT/2018/057342 discloses Pilocarpine-(R)-Lipoate and compositions and methods for the treatment of eye disorders. However, PCT/2018/057342 fails to disclose the use of physical mixture of Pilocarpine Hydrochloride and Lipoic acid [free acid] composition for the treatment of eye disorders.

Published WO document WO2018065831A1 (Intl. Appl. No. PCT/IB2017/052237) discloses Pilocarpine-(R)-Lipoate and compositions and methods for the treatment of xerostomia. However, PCT/IB2017/052237 fails to disclose the use of physical mixture of Pilocarpine Hydrochloride and Lipoic acid [free acid] composition for the treatment of xerostomia.

Thus, it is a need of the hour to provide a solution to xerostomia and dry mouth disease. The present invention provides the solution to the existing problem by providing a pharmaceutical composition comprising a physical mixture for treating or delaying the onset of xerostomia and its associated complications.

### SUMMARY OF THE INVENTION

The present disclosure relates to pharmaceutical compositions comprising a therapeutically effective amount of an antimuscarinic or an anticholinergic agent or a pharmaceutically acceptable salt or a stereoisomer thereof, in combination with a therapeutically effective amount of lipoic acid or a pharmaceutically acceptable salt or a stereoisomer or a prodrug thereof.

In certain aspects, the present disclosure provides a pharmaceutical composition comprising:
a therapeutically effective amount of an antimuscarinic or an anticholinergic agent or a pharmaceutically acceptable salt or a stereoisomer thereof; and
a therapeutically effective amount of lipoic acid or a pharmaceutically acceptable salt or a stereoisomer or a prodrug thereof.

In an aspect the antimuscarinic or anticholinergic agent is a compound of Formula I;

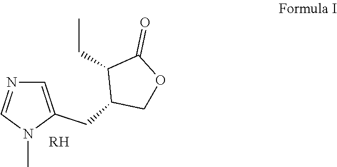

or a pharmaceutically acceptable salt or a stereoisomer thereof, wherein

### RH is

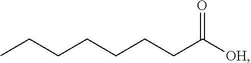

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In an aspect of the antimuscarinic or anticholinergic agent is a compound of Formula II:

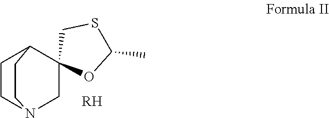

or a pharmaceutically acceptable salt or a stereoisomer thereof; wherein,

### RH is

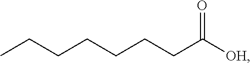

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In another aspect the antimuscarinic or anticholinergic agent is a compound of Formula III:

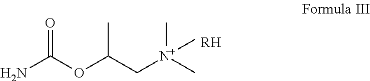

or a pharmaceutically acceptable salt or a stereoisomer thereof; wherein,

### RH is

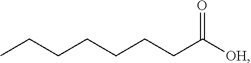

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain aspects the lipoic acid is a compound of Formula IV:

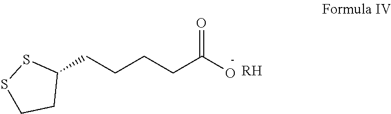

or a pharmaceutically acceptable salt or stereoisomer thereof; wherein
RH is null, H, sodium, potassium, magnesium, calcium, arginine, glutamate, lysine, glycine, proline, pyridoxine, pyridoxamine, choline, taurine, malic acid, PHMB, polyhexanide or guanidine.

In an aspect of the lipoic acid prodrug is choline ester prodrug of Formula V:

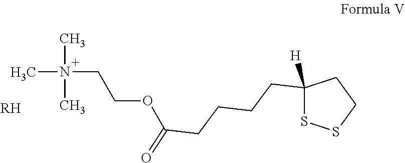

or a pharmaceutically acceptable salt or a stereoisomer thereof; wherein,
RH is H, iodine, chloride, glutamic acid, aspartic acid, lysine, ketorolac, ketoprofen, naproxen, bromine, diclofenac, nepafenac, bromfenac or glycine.

In an aspect the present disclosure provides a physical mixture comprising a therapeutically effective amount of an antimuscarinic or an anticholinergic agent or a pharmaceutically acceptable salt or a stereoisomer thereof; and a therapeutically effective amount of lipoic acid or a pharmaceutically acceptable salt or stereoisomer or prodrug thereof.

In an aspect, the present disclosure provides a physical mixture comprising a compound of Formula I and a compound of Formula IV or a compound of Formula V.

In an aspect, the present disclosure provides a physical mixture comprising a compound of Formula I and a compound of Formula IV.

In an aspect of the present disclosure, there is provided a physical mixture comprising a compound of Formula I and a compound of Formula V.

In an aspect, the present disclosure provides a physical mixture comprising a compound of Formula II and a compound of Formula IV or a compound of Formula V.

In an aspect, the present disclosure provides a physical mixture comprising a compound of Formula II and a compound of Formula IV.

In an aspect, the present disclosure provides a physical mixture comprising a compound of Formula II and a compound of Formula V.

In an aspect, the present disclosure provides a physical mixture comprising a compound of Formula III and a compound of Formula IV or a compound of Formula V.

In an aspect, the present disclosure provides a physical mixture comprising a compound of Formula III and a compound of Formula IV.

In an aspect, the present disclosure provides a physical mixture comprising a compound of Formula III and a compound of Formula V.

In an aspect of the present disclosure, there is provided a physical mixture comprising Pilocarpine HCl and R-(+)-Lipoic acid.

The disclosure also relates to a pharmaceutical composition of a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof for use in the treatment or alleviation of xerostomia, burning mouth syndrome and eye diseases or disorders.

The disclosure also relates to a pharmaceutical composition of physical mixture comprising a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof for use in the treatment or alleviation of xerostomia, burning mouth syndrome and eye diseases or disorders.

### DETAILED DESCRIPTION OF THE INVENTION

### Definitions

As used herein, the following terms and phrases shall have the meanings set forth below. Unless defined otherwise, all technical and scientific terms used herein have the same meaning as commonly understood to one of ordinary skill in the art.

The singular forms “a”, “an” and “the” encompass plural references unless the context clearly indicates otherwise.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, lngold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein, the term “metabolic condition” refers to an inborn error of metabolism (or genetic metabolic conditions) and are genetic disorders that result from a defect in one or more metabolic pathways; specifically, the function of an enzyme is affected and is either deficient or completely absent.

The term “polymorph” as used herein is art-recognized and refers to one crystal structure of a given compound.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

A “patient,” “subject,” or “host” to be treated by the subject method may mean either a human or non-human animal, such as primates, mammals, and vertebrates.

The phrase “pharmaceutically acceptable” is art-recognized. In certain embodiments, the term includes compositions, polymers and other materials and/or dosage forms which are, within the scope of sound medical judgment, suitable for use in contact with the tissues of mammals, human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “predicting” as used herein refers to assessing the probability related diseases patient will suffer from abnormalities or complication and/or terminal platelet aggregation or failure and/or death (i.e. mortality) within a defined time window (predictive window) in the future. The mortality may be caused by the central nervous system or complication. The predictive window is an interval in which the subject will develop one or more of the said complications according to the predicted probability. The predictive window may be the entire remaining lifespan of the subject upon analysis by the method of the present invention.

As used herein, the term “subject,” that is interchangeable with “patient” or “host”, refers to an animal, preferably a mammal, and most preferably a human. Subjects include primates and other mammals such as equines, cattle, swine and sheep; and poultry and pets in general.

As used herein, the term “stereoisomer” is a term used for all isomers of individual compounds of Formula I, Formula II, Formula III, Formula IV or Formula V that differs only in the orientation of their atoms in space. The term stereoisomer includes mirror image isomers (enantiomers) of compounds of Formula I, Formula II, Formula III, Formula IV or Formula V, mixtures of mirror image isomers (racemates, racemic mixtures) of compounds of Formula I, Formula II, Formula III, Formula IV or Formula V, geometric (cis/trans or E/Z, R/S) isomers of compounds of Formula I, Formula II, Formula III, Formula IV or Formula V and isomers of compounds of Formula I, Formula II, Formula III, Formula IV or Formula V with more than one chiral center that are not mirror images of one another (diastereoisomers).

The term “treating” is art recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, and includes administration of a composition which reduces the frequency of, or delays the onset of, symptoms of a medical condition in a subject relative to a subject which does not receive the composition.

The phrase “therapeutically effective amount” is an art-recognized term. In certain embodiments, the term refers to an amount of a solvate or hydrate or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular composition being administered, the size of the subject, or the severity of the disease or condition. One of ordinary skill in the art may empirically determine the effective amount of a particular composition without undue experimentation.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present disclosure include such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present disclosure.

In certain embodiments, the pharmaceutical compositions described herein are formulated in a manner such that said compositions will be delivered to a patient in a therapeutically effective amount, as part of a prophylactic or therapeutic treatment. The desired amount of the composition to be administered to a patient will depend on absorption, inactivation, and excretion rates of the drug as well as the delivery rate of the hydrates or solvates and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular solvate or hydrate or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active solvate or hydrate and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of a composition for the disease being treated, even if the agents comprised in the composition are subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrases “Physical mixture” refers to a mixture in which the constituent substances are not chemically combined though they may be so intimately mingled as to be impossible to separate by simple mechanical means.

In an embodiment of the present disclosure, there is provided a pharmaceutical composition comprising a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof.

The present disclosure also contemplates prodrugs of the compounds comprised in the compositions as disclosed herein, as well as pharmaceutically acceptable hydrates or solvates of said prodrugs.

In certain embodiments, the antimuscarinic or anticholinergic agent is selected from the group consisting of pilocarpine, cevimeline and bethanechol, or a pharmaceutically acceptable salt or a stereoisomer thereof. In further embodiments, the antimuscarinic or anticholinergic agent is pilocarpine, or a pharmaceutically acceptable salt or a stereoisomer thereof. In other embodiments, the antimuscarinic or anticholinergic agent is cevimeline, or a pharmaceutically acceptable salt or a stereoisomer thereof. In a further embodiment, the antimuscarinic or anticholinergic agent is bethanechol, or a pharmaceutically acceptable salt or a stereoisomer thereof.

In certain embodiments, the antimuscarinic or anticholinergic agent is a compound of Formula I:

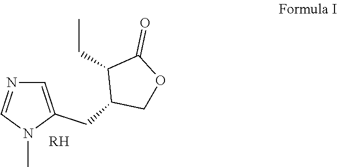

or a pharmaceutically acceptable salt or a stereoisomer thereof; wherein,

### RH is

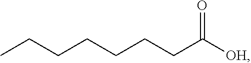

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of hydrates or solvates of pilocarpine and an acidic moiety containing compound selected from RH in which the pilocarpine is protonated, and the acid moiety RH of the pharmaceutically acceptable salt is at least in partially ionic form. In some instances, however, for example depending on the pH of the environment, the composition may be in the form of a mixture of pilocarpine and acid components RH.

In certain embodiments, the antimuscarinic or anticholinergic agent is a compound of Formula II:

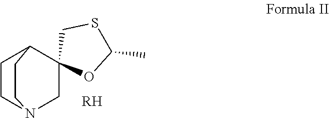

or a pharmaceutically acceptable salt or a stereoisomer thereof; wherein,

### RH is

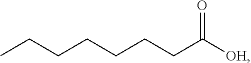

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of salts of cevimeline and an acid moiety containing compound selected from RH in which the cevimeline is in protonated form and the acid moiety RH is at least in partially ionic form. In some instances, however, for example depending on the pH of the environment, the composition may be in the form of a mixture of cevimeline and an acid moiety RH. In further embodiments, the compositions disclosed herein may further comprise a pharmaceutically acceptable carrier, diluent, or excipient, or a combination thereof.

In certain embodiments, the antimuscarinic or anticholinergic agent is a compound of Formula III:

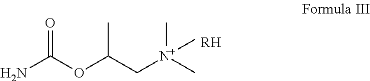

or a pharmaceutically acceptable salt or a stereoisomer thereof; wherein,

### RH is

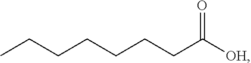

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of salts of bethanechol and an acid moiety containing compound selected from RH in which the bethanechol is in protonated form and the acid moiety RH is at least in partially ionic form. In some instances, however, for example depending on pH of the environment, the composition may be in the form of a mixture of bethanechol and an acid moiety RH. In further embodiments, the compositions disclosed herein may further comprise a pharmaceutically acceptable carrier, diluent, or excipient, or a combination thereof.

In certain embodiments, the lipoic acid is (R)-(+)-lipoic acid (RLA) or (S)-(−)-lipoic acid (SLA) or a racemic mixture (R/S)-lipoic acid (R/S-LA).

In certain embodiments, the lipoic acid is a compound of Formula IV:

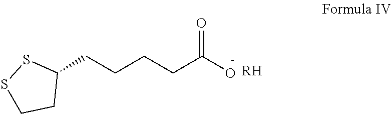

or a pharmaceutically acceptable salt or a stereoisomer thereof; wherein,
RH is null, H, sodium, potassium, magnesium, calcium, arginine, glutamate, lysine, glycine, proline, pyridoxine, pyridoxamine, choline, taurine, malic acid, PHMB, polyhexanide or guanidine.

It is contemplated that when a particular compound is mentioned by name, for example, pilocarpine, cevimeline, or bethanechol, the scope of the present disclosure encompasses pharmaceutically acceptable salts, esters, amides, or prodrugs of the named compound. Further, when the named compound comprises a chiral center the scope of the present disclosure also includes compositions comprising the racemic mixture of the two enantiomers, as well as compositions comprising each enantiomer individually substantially free of the other enantiomer. In further embodiments, if the named compound comprises more than one chiral center, the scope of the present disclosure also includes compositions comprising a mixture of the various diastereomers, as well as compositions comprising each diastereomer substantially free of the other diastereomers. Further, for example, commercially available pilocarpine comprises two stereocenters. The scope of the present disclosure includes pharmaceutical compositions comprising all four diastereomers, pharmaceutical compositions comprising the racemic mixture of R,R and S,S isomers, pharmaceutical compositions comprising the racemic mixture of R,S and S,R isomers, pharmaceutical compositions comprising the R,R enantiomer substantially free of the other diastereomers, pharmaceutical compositions comprising the S,S enantiomer substantially free of the other diastereomers, pharmaceutical compositions comprising the R,S enantiomer substantially free of the other diastereomers, and pharmaceutical compositions comprising the S,R enantiomer substantially free of the other diastereomers.

In certain embodiments, the lipoic acid prodrug is a choline ester prodrug compound of Formula V:

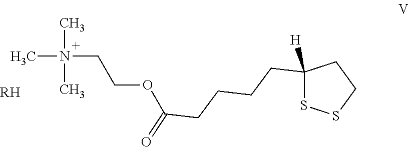

or a pharmaceutically acceptable salt or a stereoisomer thereof; wherein,
RH is H, chloride, iodine, glutamic acid, aspartic acid, lysine, ketorolac, ketoprofen, naproxen, bromine, diclofenac, nepafenac, bromfenac or glycine.

It is to be contemplated that when a particular compound is mentioned by name, for example, pilocarpine, cevimeline, or bethanechol, the scope of the present disclosure encompasses pharmaceutically acceptable salts, esters, amides, or prodrugs of the named compound. Further, when the named compound comprises a chiral center the scope of the present disclosure also includes compositions comprising the racemic mixture of the two enantiomers, as well as compositions comprising each enantiomer individually substantially free of the other enantiomer. In further embodiments, if the named compound comprises more than one chiral center, the scope of the present disclosure also includes compositions comprising a mixture of the various diastereomers, as well as compositions comprising each diastereomer substantially free of the other diastereomers. Further, for example, commercially available pilocarpine comprises two stereocenters. The scope of the present disclosure includes pharmaceutical compositions comprising all four diastereomers, pharmaceutical compositions comprising the racemic mixture of R,R and S,S isomers, pharmaceutical compositions comprising the racemic mixture of R,S and S,R isomers, pharmaceutical compositions comprising the R,R enantiomer substantially free of the other diastereomers, pharmaceutical compositions comprising the S,S enantiomer substantially free of the other diastereomers, pharmaceutical compositions comprising the R,S enantiomer substantially free of the other diastereomers, and pharmaceutical compositions comprising the S,R enantiomer substantially free of the other diastereomers.

In an embodiment the present disclosure provides a physical mixture comprising a therapeutically effective amount of an antimuscarinic or an anticholinergic agent or a pharmaceutically acceptable salt or a stereoisomer thereof; and a therapeutically effective amount of lipoic acid or a pharmaceutically acceptable salt or stereoisomer or prodrug thereof.

In an embodiment the present disclosure provides a physical mixture comprising a compound of Formula I and a compound of Formula IV or a compound of Formula V.

In an embodiment, the present disclosure provides a physical mixture comprising a compound of Formula I and a compound of Formula IV.

In an embodiment, the present disclosure provides a physical mixture comprising a compound of Formula II and a compound of Formula IV or a compound of Formula V.

In an embodiment, the present disclosure provides a physical mixture comprising a compound of Formula II and a compound of Formula IV.

In an embodiment, the present disclosure provides a physical mixture comprising a compound of Formula II and a compound of Formula V.

In an embodiment, the present disclosure provides a physical mixture comprising a compound of Formula III and a compound of Formula IV or a compound of Formula V.

In an embodiment, the present disclosure provides a physical mixture comprising a compound of Formula III and a compound of Formula IV.

In an embodiment, the present disclosure provides a physical mixture comprising a compound of Formula III and a compound of Formula V. In an embodiment of the present disclosure, there is provided a pharmaceutical composition comprising the compound of Formula I present in a therapeutically effective dose range of about 0.001 mg to about 200 mg and the compound of Formula IV present in a therapeutically effective dose range from about 5 mg to about 4 g wherein the compound of Formula I and the compound of Formula IV are included individually or as physical mixture thereof.

In certain embodiments, the present disclosure relates to a pharmaceutical composition comprising a compound of Formula I in combination with a compound of Formula IV included individually or as a physical mixture thereof. In yet other embodiments, the present disclosure relates to a composition comprising pilocarpine in combination with lipoic acid. In further embodiments, the pharmaceutical composition comprises pilocarpine in combination with R-lipoic acid. In further embodiments, the pharmaceutical composition comprises pilocarpine hydrochloride in combination with racemic lipoic acid. In other embodiments, the pharmaceutical composition comprises pilocarpine hydrochloride in combination with R-lipoic acid.

In an embodiment, the present disclosure provides a physical mixture comprising a compound of Formula II and a compound of Formula IV.

In certain embodiments, the present disclosure relates to a pharmaceutical composition comprising a compound of Formula II in combination with a compound of Formula IV included individually or as a physical mixture thereof. In yet other embodiments, the present disclosure relates to a composition comprising cevimeline in combination with lipoic acid. In further embodiments, the pharmaceutical composition comprises cevimeline in combination with R-lipoic acid. In further embodiments, the pharmaceutical composition comprises cevimeline hydrochloride in combination with racemic lipoic acid. In other embodiments, the pharmaceutical composition comprises cevimeline hydrochloride in combination with R-lipoic acid.

In an embodiment, the present disclosure provides a physical mixture comprising a compound of Formula III and a compound of Formula IV.

In yet further embodiments, the present disclosure relates to a pharmaceutical composition comprising a compound of Formula III in combination with a compound of Formula IV included individually or as a physical mixture thereof. In yet other embodiments, the present disclosure relates to a composition comprising bethanechol in combination with lipoic acid. In further embodiments, the pharmaceutical composition comprises bethanechol in combination with R-lipoic acid. In yet other embodiments, the pharmaceutical composition comprises bethanechol hydrochloride in combination with racemic lipoic acid. In other embodiments, the pharmaceutical composition comprises bethanechol hydrochloride in combination with R-lipoic acid.

In an embodiment of the present disclosure, there is provided a pharmaceutical composition comprising: a compound of Formula I, a compound of Formula V and at least one pharmaceutically acceptable excipient.

In an embodiment, the present disclosure provides a physical mixture comprising a compound of Formula I and a compound of Formula V.

In certain embodiments, the present disclosure relates to a pharmaceutical composition comprising a compound of Formula I in combination with a compound of Formula V included individually or as a physical mixture thereof. In yet other embodiments, the present disclosure relates to a composition comprising pilocarpine in combination with lipoic acid. In further embodiments, the pharmaceutical composition comprises pilocarpine in combination with R-lipoic acid. In further embodiments, the pharmaceutical composition comprises pilocarpine hydrochloride in combination with racemic lipoic acid. In other embodiments, the pharmaceutical composition comprises pilocarpine hydrochloride in combination with R-lipoic acid.

In an embodiment of the present disclosure, there is provided a physical mixture comprising Pilocarpine HCl and R-(+)-Lipoic acid.

In an embodiment of the present disclosure, there is provided a pharmaceutical composition comprising the compound of Formula I present in a therapeutically effective dose range of about 0.001 mg to about 200 mg and the compound of Formula V present in a therapeutically effective dose range from about 5 mg to about 4 g wherein the compound of Formula I and the compound of Formula V are included individually or as physical mixture thereof.

In certain embodiments, the composition comprising R enantiomer is substantially free of S enantiomer, or a composition comprising S enantiomer is substantially free of R enantiomer. In this context, “substantially free” means, the composition comprises less than about 20%, or less than about 15%, or less than about 10%, or less than about 5%, or less than about 3% or less than about 1% of the minor enantiomer.

In an embodiment, the present disclosure provides a physical mixture comprising a compound of Formula II and a compound of Formula V.

In certain embodiments, the present disclosure relates to a pharmaceutical composition comprising a compound of Formula II in combination with a compound of Formula V included individually or as a physical mixture thereof. In yet other embodiments, the present disclosure relates to a composition comprising cevimeline in combination with lipoic acid. In further embodiments, the pharmaceutical composition comprises cevimeline in combination with R-lipoic acid. In further embodiments, the pharmaceutical composition comprises cevimeline hydrochloride in combination with racemic lipoic acid. In other embodiments, the pharmaceutical composition comprises cevimeline hydrochloride in combination with R-lipoic acid.

In an embodiment, the present disclosure provides a physical mixture comprising a compound of Formula III and a compound of Formula V.

In yet further embodiments, the present disclosure relates to a pharmaceutical composition comprising a compound of Formula III in combination with a compound of Formula V included individually or as a physical mixture thereof. In yet other embodiments, the present disclosure relates to a composition comprising bethanechol in combination with lipoic acid. In further embodiments, the pharmaceutical composition comprises bethanechol in combination with R-lipoic acid. In yet other embodiments, the pharmaceutical composition comprises bethanechol hydrochloride in combination with racemic lipoic acid. In other embodiments, the pharmaceutical composition comprises bethanechol hydrochloride in combination with R-lipoic acid.

In further embodiments, the compositions disclosed herein may further comprise a pharmaceutically acceptable carrier, diluent, or excipient, or a combination thereof.

This application also discloses a pharmaceutical composition comprising the compound of Formula I, II, or III; the compound of Formula IV or V; or physical mixture thereof; and a pharmaceutically acceptable carrier. The pharmaceutical composition of the present disclosure may be formulated into dosage form for dermal, ocular, systemic or topical or oral administration. The pharmaceutical composition may be also formulated into dosage form for oral administration, oral solution, dermal, cream, gels, ocular, injection, subdermal administration, or transdermal administration. The pharmaceutical composition may further comprise at least one of a pharmaceutically acceptable stabilizer, diluent, surfactant, filler, binder, and lubricant.

In many embodiments, the pharmaceutical compositions described herein will incorporate the disclosed compound of Formula I, II, or III, and compound of Formula IV or V, to be delivered in an amount sufficient to deliver to a patient a therapeutically effective amount of said compound, or composition as part of a prophylactic or therapeutic treatment. The desired concentration of formula I or its pharmaceutical acceptable hydrates or solvates will depend on absorption, inactivation, and excretion rates of the drug as well as the delivery rate of the hydrates or solvates from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The compositions as disclosed herein can be used as a medicament. In certain embodiments, the compositions are particularly useful in treatment or alleviation of xerostomia, burning mouth syndrome and eye diseases or disorders. In certain embodiments, the compositions are useful in the treatment or alleviation of xerostomia or its related complications. The compositions for example, useful in treating a subject suffering from xerostomia or its related complications manifested from metabolic or genetic conditions or disorders, metabolic diseases, chronic diseases or disorders; neurodegenerative disorders, metabolic condition, hepatology, cancer, respiratory, hematological, orthopedic, cardiovascular, renal, skin, vascular or ocular complications.

In further embodiments, the compositions as disclosed herein are useful in the treatment or alleviation of burning mouth syndrome.

In further embodiments, the compositions as disclosed herein are useful in the treatment or alleviation of one or more eye diseases or disorders; wherein the eye disease or disorder is selected from the group consisting of presbyopia, retinal arterial occlusions, (in particular central retinal artery occlusion), age related visual degradation (near and far visual acuity; visual field), diabetic retinopathy, retinal vein occlusion (in particular central retinal vein occlusion or branch retinal vein occlusion), visual degradation of visual acuity and visual field, exudative macular degeneration (age related macular degeneration, high myopia; macular degeneration), myopia, macular oedema, central serious chorio-retinopathy, papillitis, uveitis, glaucoma and/or glaucomatous neuropathy. In yet other embodiments, the compositions disclosed herein are useful in the treatment or alleviation of presbyopia, glaucoma and/or glaucomatous neuropathy.

In certain embodiments, the disclosure also provides a kit comprising any of the pharmaceutical compositions disclosed herein. The kit may comprise instructions for use in the treatment of xerostomia or its related complications, burning mouth syndrome or eye diseases or disorders.

In further embodiments, the present disclosure also relates to a method of treating xerostomia in a subject comprising administering to a subject in need thereof a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof; wherein the antimuscarinic or an anticholinergic agent and lipoic acid are as described above. In certain embodiments, the subject is a mammal such as a human, or a non-human mammal. In further embodiments, the subject is a human.

In yet other embodiments, the present disclosure also relates to a method of treating burning mouth syndrome in a subject comprising administering to a subject in need thereof a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof; wherein the antimuscarinic or an anticholinergic agent and lipoic acid are as described above. In certain embodiments, the subject is a mammal such as a human, or a non-human mammal. In further embodiments, the subject is a human.

The present disclosure also relates to a method of treating one or more eye diseases or disorders in a subject comprising administering to a subject in need thereof a therapeutically effective amount of an antimuscarinic or an anticholinergic agent or a pharmaceutically acceptable salt or a stereoisomer thereof in combination with a therapeutically effective amount of lipoic acid or a pharmaceutically acceptable salt or a stereoisomer or a prodrug thereof; wherein the antimuscarinic or an anticholinergic agent and lipoic acid are as described above, and the eye disease or disorder is selected from the group consisting of presbyopia, retinal arterial occlusions, (in particular central retinal artery occlusion), age related visual degradation (near and far visual acuity; visual field), diabetic retinopathy, retinal vein occlusion (in particular central retinal vein occlusion or branch retinal vein occlusion), visual degradation of visual acuity and visual field, exudative macular degeneration (age related macular degeneration, high myopia; macular degeneration), myopia, macular oedema, central serious chorio-retinopathy, papillitis, uveitis, glaucoma and/or glaucomatous neuropathy. In further embodiments, the eye disease or disorder is presbyopia, glaucoma or glaucomatous neuropathy. In yet other embodiments, the subject is a mammal such as a human, or a non-human mammal. In further embodiments, the subject is a human.

In certain embodiments, the antimuscarinic or an anticholinergic agent and lipoic acid may be administered simultaneously or separately. In further embodiments, the antimuscarinic or an anticholinergic agent may be administered prior to the lipoic acid. In yet other embodiments, the antimuscarinic or an anticholinergic may be administered subsequent to the lipoic acid. In some embodiments, when the antimuscarinic or an anticholinergic agent and lipoic acid may be administered within one dosage form. In further embodiments, when the antimuscarinic or an anticholinergic agent and lipoic acid may be administered within different dosage forms.

In certain embodiments of the compositions, the antimuscarinic or an anticholinergic agent, or a pharmaceutically acceptable salt or a stereoisomer thereof, may present in a dose ranging from about 0.01 mg to about 50 mg. In further embodiments, the antimuscarinic or an anticholinergic agent is present in a dose of about 0.01 mg to about 40 mg. In other embodiments, the antimuscarinic or an anticholinergic agent or a pharmaceutically acceptable salt or a stereoisomer thereof, is present in a dose of about 1 g to about 20 g. In yet other embodiments, the antimuscarinic or an anticholinergic agent or a pharmaceutically acceptable salt or a stereoisomer thereof, is present in a dose of about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In further embodiments, the lipoic acid or a pharmaceutically acceptable salt or a stereoisomer or a prodrug thereof, may be present in a dose of from about 5 mg to about 4 g. In further embodiments, the lipoic acid or a pharmaceutically acceptable salt or a stereoisomer thereof is present in a dose of about 10 mg to about 2 g or about 100 mg to about 1.5 g. In yet other embodiment, the lipoic acid or a pharmaceutically acceptable salt or a stereoisomer or a prodrug thereof is present in a dose of about 500 mg to about 1 g.

The application also discloses a pharmaceutical composition comprising a pharmaceutically acceptable carrier and any of the compositions described herein. In certain embodiments, the pharmaceutical composition is formulated for systemic administration, oral administration, parenteral administration, subdermal administration, or transdermal administration as oral solution, oral rinsing solution, oral antiseptic solution, oral mucoadhesive spray, lozenge, buccal tablet, hard gelatin mouth dissolving tablet, effervescent tablet, mouth dissolving tablet, hydrogel, sustained release tablet, injection, paste, cream, lotion, gel, or the like.

In certain embodiments, the active ingredients of the combination of the present disclosure can be administered by same or different route of administration. For example, the antimuscarinic or an anticholinergic agent of the present disclosure can be administered orally, and the lipoic acid can be administered transdermally.

In certain embodiments, the pharmaceutical compositions described herein are formulated in a manner such that said compositions will be delivered to a subject in a therapeutically effective amount, as part of a prophylactic or therapeutic treatment. The desired amount of the composition to be administered to a subject will depend on absorption, inactivation, and excretion rates of the drug as well as the delivery rate of the hydrates or solvates and compositions from the subject formulations. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular solvate or hydrate or composition may be adjusted to accommodate variations in the treatment parameters. Such treatment parameters include the clinical use to which the preparation is put, e.g., the site treated, the type of subject, e.g., human or non-human, adult or child, and the nature of the disease or condition.

In certain embodiments, the dosage of the subject compositions provided herein may be determined by reference to the plasma concentrations of the therapeutic composition or other encapsulated materials. For example, the maximum plasma concentration (Cmax) and the area under the plasma concentration-time curve from time 0 to infinity may be used.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., a therapeutic and/or biologically active solvate or hydrate and/or composition, for a sustained or extended period (as compared to the release from a bolus). This release may result in prolonged delivery of therapeutically effective amounts of any of the therapeutic agents disclosed herein.

Generally, in carrying out the methods detailed in this application, an effective dose of the compounds disclosed herein is in the range of about 0.01 mg/kg/day to about 100 mg/kg/day in single or divided doses, for instance about 0.01 mg/kg/day to about 50 mg/kg/day in single or divided doses. The compounds may be administered at a dose of, for example, less than about 0.2 mg/kg/day, 0.5 mg/kg/day, 1.0 mg/kg/day, 5 mg/kg/day, 10 mg/kg/day, 20 mg/kg/day, 30 mg/kg/day, or 40 mg/kg/day.

Compounds may also be administered to a human subject at a dose of, for example, between about 0.1 mg and about 1000 mg, between about 5 mg and about 80 mg, or less than about 1.0 mg, 9.0 mg, 12.0 mg, 20.0 mg, 50.0 mg, 75.0 mg, 100 mg, 300 mg, 400 mg, 500 mg, 800 mg, 1000 mg, 2000 mg, or 5000 mg per day.

In certain embodiments, the compositions herein are administered at an amount that is less than about 95%, 90%, 80%, 70%, 60%, 50%, 40%, 30%, 20%, or 10% of the compound as disclosed herein required for the same therapeutic benefit.

In certain embodiments of the compositions, the antimuscarinic or an anticholinergic agent, or a pharmaceutically acceptable salt or a stereoisomer thereof, may be present in a dose of from about 0.01 mg to about 50 mg. In further embodiments, the antimuscarinic or an anticholinergic agent is present in a dose of about 0.01 mg to about 40 mg. For example, the antimuscarinic or an anticholinergic agent selected from pilocarpine, cevimeline and bethanechol or a pharmaceutically acceptable salt or a stereoisomer thereof, is present in a dose of about 1 g to about 20 g.

In yet other embodiments, the antimuscarinic or an anticholinergic agent or a pharmaceutically acceptable salt or a stereoisomer thereof, is present in a dose of about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

An effective amount may be sufficient to prohibit, treat, alleviate, ameliorate, halt, restrain, slow or reverse the progression, or reduce the severity of a complication resulting from nerve damage or demyelization and/or elevated reactive oxidative-nitrosative species and/or abnormalities in neurotransmitter homeostasis's, in subjects who are at risk for such complications. As such, these methods include both medical therapeutic (acute) and/or prophylactic (prevention) administration as appropriate. The amount and timing of compositions administered will, of course, be dependent on the subject being treated, on the severity of the affliction, on the manner of administration and on the judgment of the prescribing physician. Thus, because of subject-to-subject variability, the dosages given above are a guideline and the physician may titrate doses of the drug to achieve the treatment that the physician considers appropriate for the subject. In considering the degree of treatment desired, the physician must balance a variety of factors such as age of the subject, presence of preexisting disease, as well as presence of other diseases.

The compositions provided by this application may be administered to a subject in need of treatment by a variety of conventional routes of administration, including orally, topically, parenterally, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

The compositions may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical formulations formed by combining the compositions and the pharmaceutically acceptable carriers, vehicles or diluents are then readily administered in a variety of dosage forms such as tablets, powders, lozenges, sterile eye solution, ocular solution, syrups, injectable solutions and the like. These pharmaceutical compositions can, if desired, contain additional ingredients such as flavorings, binders, excipients and the like. Thus, for purposes of oral administration, tablets containing various excipients such as L-arginine, sodium citrate, calcium carbonate and calcium phosphate may be employed along with various disintegrates such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Appropriate materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof. The compounds as disclosed herein may also be enterically coated comprising of various excipients, as is well known in the pharmaceutical art.

For parenteral administration, solutions of the compositions may be prepared in (for example) sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

Generally, a composition as described herein may be administered orally, or parenterally for example intravenous, intramuscular, subcutaneous or intramedullary. Topical administration may also be indicated, for example, where the subject is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

Illustratively, dosage levels of the administered active ingredients are: intravenous, about 0.1 mg/kg to about 200 mg/kg; intramuscular, about 1 mg/kg to about 500 mg/kg; orally, about 5 mg/kg to about 1000 mg/kg; intranasal instillation, about 5 mg/kg to about 1000 mg/kg; and aerosol, about 5 mg/kg to about 1000 mg/kg of host body weight.

Expressed in terms of concentration, an active ingredient can be present in the compositions of the present invention for localized use about the cutis, intranasally, pharyngolaryngeally, bronchially, intravaginally, rectally, or ocularly in a concentration of from about 0.01% w/w to about 50% w/w of the composition; preferably about 1% w/w to about 20% w/w of the composition; and for parenteral use in a concentration of from about 0.05% w/w to about 50% w/v of the composition and preferably from about 5% w/w to about 20% w/v.

The compositions of the present disclosure are preferably presented for administration to humans and animals in unit dosage forms, such as tablets, capsules, pills, powders, sterile ocular solution, sterile eye solution, ocular implant mediated delivery, granules, suppositories, sterile parenteral solutions or suspensions, sterile non-parenteral solutions of suspensions, and oral solutions or suspensions and the like, containing suitable quantities of an active ingredient. For oral administration either solid or fluid unit dosage forms can be prepared. For ocular administration either sterile solution or device or implant mediated delivery unit dosage forms can be prepared.

In certain embodiments, the tablet core contains one or more hydrophilic polymers. Suitable hydrophilic polymers include, but are not limited to, water swellable cellulose derivatives, polyalkylene glycols, thermoplastic polyalkylene oxides, acrylic polymers, hydrocolloids, clays, gelling starches, swelling cross-linked polymers, and mixtures thereof. Examples of suitable water swellable cellulose derivatives include, but are not limited to, sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose, and mixtures thereof. Examples of suitable polyalkylene glycols include, but are not limited to, polyethylene glycol. Examples of suitable thermoplastic polyalkylene oxides include, but are not limited to, poly(ethylene oxide). Examples of suitable acrylic polymers include, but are not limited to, potassium methacrylatedivinylbenzene copolymer, polymethylmethacrylate, high-molecular weight crosslinked acrylic acid homopolymers and copolymers such as those commercially available from Noveon Chemicals under the tradename CARBOPOL™. Examples of suitable hydrocolloids include, but are not limited to, alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan, and mixtures thereof. Examples of suitable clays include, but are not limited to, smectites such as bentonite, kaolin, and laponite; magnesium trisilicate; magnesium aluminum silicate; and mixtures thereof. Examples of suitable gelling starches include, but are not limited to, acid hydrolyzed starches, swelling starches such as sodium starch glycolate and derivatives thereof, and mixtures thereof. Examples of suitable swelling cross-linked polymers include, but are not limited to, cross-linked polyvinyl pyrrolidone, cross-linked agar, and cross-linked carboxymethylcellulose sodium, and mixtures thereof.

The carrier may contain one or more suitable excipients for the formulation of tablets. Examples of suitable excipients include, but are not limited to, fillers, adsorbents, binders, disintegrants, lubricants, glidants, release-modifying excipients, superdisintegrants, antioxidants, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their hydrates or solvates, such as magnesium stearate and stearic acid, talc, glycerides waxes, and mixtures thereof. Suitable glidants include, but are not limited to, colloidal silicon dioxide. Suitable release-modifying excipients include, but are not limited to, insoluble edible materials, pH-dependent polymers, and mixtures thereof.

Suitable insoluble edible materials for use as release-modifying excipients include, but are not limited to, water-insoluble polymers and low-melting hydrophobic materials, copolymers thereof, and mixtures thereof. Examples of suitable water-insoluble polymers include, but are not limited to, ethylcellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their hydrates or solvates, and mixtures thereof. Examples of suitable fatty acid esters include, but are not limited to, sucrose fatty acid esters, mono-, di-, and triglycerides, glyceryl behenate, glyceryl palmitostearate, glycerylmonostearate, glyceryltristearate, glyceryltrilaurylate, glycerylmyristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidylenositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof. Examples of super disintegrants include, but are not limited to, croscarmellose sodium, sodium starch glycolate and cross-linked povidone (crospovidone). In certain embodiments, the tablet core contains up to about 5 percent by weight of such super disintegrant.

Examples of antioxidants include, but are not limited to, tocopherols, ascorbic acid, sodium pyrosulfite, butylhydroxytoluene, butylatedhydroxyanisole, edetic acid, and edetate hydrates or solvates, and mixtures thereof. Examples of preservatives include, but are not limited to, citric acid, tartaric acid, lactic acid, malic acid, acetic acid, benzoic acid, and sorbic acid, and mixtures thereof.

In certain embodiments, the immediate release coating has an average thickness of at least 50 microns, such as from about 50 microns to about 2500 microns; e.g., from about 250 microns to about 1000 microns. In an embodiment, the immediate release coating is typically compressed at a density of more than about 0.9 g/cc, as measured by the weight and volume of that specific layer.

In certain embodiments, the immediate release coating contains a first portion and a second portion, wherein at least one of the portions contains the second pharmaceutically active agent. In certain embodiments, the portions contact each other at a center axis of the tablet. In certain embodiments, the first portion includes the first pharmaceutically active agent and the second portion includes the second pharmaceutically active agent.

In certain embodiments, the first portion contains the first pharmaceutically active agent and the second portion contains the second pharmaceutically active agent. In certain embodiments, one of the portions contains a third pharmaceutically active agent. In certain embodiments one of the portions contains a second immediate release portion of the same pharmaceutically active agent as that contained in the tablet core.

In certain embodiments, the outer coating portion is prepared as a dry blend of materials prior to addition to the coated tablet core. In another embodiment the outer coating portion is included of a dried granulation including the pharmaceutically active agent.

Formulations with different drug release mechanisms described above could be combined in a final dosage form containing single or multiple units. Examples of multiple units include multilayer tablets, capsules containing tablets, beads, or granules in a solid or liquid form. Typical, immediate release formulations include compressed tablets, gels, films, coatings, liquids and particles that can be encapsulated, for example, in a gelatin capsule. Many methods for preparing coatings, covering or incorporating drugs, are known in the art.

The immediate release dosage, unit of the dosage form, i.e., a tablet, a plurality of drug-containing beads, granules or particles, or an outer layer of a coated core dosage form, contains a therapeutically effective quantity of the active agent with conventional pharmaceutical excipients. The immediate release dosage unit may or may not be coated and may or may not be admixed with the delayed release dosage unit or units as in an encapsulated mixture of immediate release drug-containing granules, particles or beads and delayed release drug-containing granules or beads.

Extended release formulations are generally prepared as diffusion or osmotic systems, for example, as described in “Remington—The Science and Practice of Pharmacy”, 20th. Ed., Lippincott Williams & Wilkins, Baltimore, Md., 2000). A diffusion system typically consists of one of two types of devices, reservoir and matrix, which are well known and described in die art. The matrix devices are generally prepared by compressing the drug with a slowly dissolving polymer carrier into a tablet form.

An immediate release portion can be added to the extended release system by means of either applying an immediate release layer on top of the extended release core; using coating or compression processes or in a multiple unit system such as a capsule containing extended and immediate release beads.

Delayed release dosage formulations are created by coating a solid dosage form with a film of a polymer which is insoluble in the acid environment of the stomach, but soluble in the neutral environment of small intestines. The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule.

A pulsed release dosage form is one that mimics a multiple dosing profile without repeated dosing and typically allows at least a twofold reduction in dosing frequency as compared to the drug presented as a conventional dosage form (e.g., as a solution or prompt drug-releasing, conventional solid dosage form). A pulsed release profile is characterized by a time period of no release (lag time) or reduced release followed by rapid drug release.

Each dosage form contains a therapeutically effective amount of active agent. In certain embodiments of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 3.0 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of beads, granules particles that release drug substantially immediately following oral administration to provide an initial dose. The delayed release dosage unit contains a plurality of coated beads or granules, which release drug approximately 3 hours to 14 hours following oral administration to provide a second dose.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more compounds of Formula I, Formula II, Formula III, Formula IV or Formula V, and/or other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In addition, in certain embodiments, subject compositions of the present application maybe lyophilized or subjected to another appropriate drying technique such as spray drying. The subject compositions may be administered once or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

Methods of preparing these formulations or compositions include the step of bringing into association subject compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

The compounds of Formula I, Formula II, Formula III, Formula IV or Formula V described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

Liquid dosage forms for oral administration include pharmaceutically acceptable emulsions, micro emulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylatedisostearyl alcohols, polyoxyethylene sorbitol, and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

Dosage forms for transdermal administration include powders, sprays, ointments, pastes, creams, lotions, gels, solutions, patches, and inhalants. A subject composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to subject compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a subject composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

Methods of delivering a composition or compositions via a transdermal patch are known in the art. Exemplary patches and methods of patch delivery are described in U.S. Pat. Nos. 6,974,588, 6,564,093, 6,312,716, 6,440,454, 6,267,983, 6,239,180, and 6,103,275.

In another embodiment, a transdermal patch may comprise: a substrate sheet comprising a composite film formed of a resin composition comprising 100 parts by weight of a polyvinyl chloride-polyurethane composite and 2-10 parts by weight of a styrene-ethylene-butylene-styrene copolymer, a first adhesive layer on the one side of the composite film, and a polyalkylene terephthalate film adhered to the one side of the composite film by means of the first adhesive layer, a primer layer which comprises a saturated polyester resin and is formed on the surface of the polyalkylene terephthalate film; and a second adhesive layer comprising a styrene-diene-styrene block copolymer containing a pharmaceutical agent layered on the primer layer. A method for the manufacture of the above-mentioned substrate sheet comprises preparing the above resin composition molding the resin composition into a composite film by a calendar process, and then adhering a polyalkylene terephthalate film on one side of the composite film by means of an adhesive layer thereby forming the substrate sheet and forming a primer layer comprising a saturated polyester resin on the outer surface of the polyalkylene terephthalate film.

Another type of patch comprises incorporating the drug directly in a pharmaceutically acceptable adhesive and laminating the drug-containing adhesive onto a suitable backing member, e.g. a polyester backing membrane. The drug should be present at a concentration which will not affect the adhesive properties, and at the same time deliver the required clinical dose.

Transdermal patches may be passive or active. Passive transdermal drug delivery systems currently available, such as the nicotine, estrogen and nitroglycerine patches, deliver small-molecule drugs. Many of the newly developed proteins and peptide drugs are too large to be delivered through passive transdermal patches and may be delivered using technology such as electrical assist (iontophoresis) for large-molecule drugs.

Iontophoresis is a technique employed for enhancing the flux of ionized substances through membranes by application of electric current. One example of an iontophoretic membrane is given in U.S. Pat. No. 5,080,646. The principal mechanisms by which iontophoresis enhances molecular transport across the skin are (a) repelling a charged ion from an electrode of the same charge, (b) electro-osmosis, the convective movement of solvent that occurs through a charged pore in response the preferential passage of counter-ions when an electric field is applied or (c) increase skin permeability due to application of electrical current.

Certain ranges are disclosed herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning and the meaning of such terms is independent at each occurrence thereof and is as commonly understood by one of skill in art to which the subject matter herein belongs.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present disclosure include such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present disclosure.

### Examples

The disclosure will now be illustrated with working examples, which are intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

Pilocarpine HCl (101.5 mg) and R-(+)-Lipoic acid (85.8 mg) were separately weighed, both the chemicals were transferred into a 50 ml flask and mixed well on rotavac (without vacuum at room temperature) under stirring for 10 min. This mixture was then transferred into a mortor and thoroughly ground. The DSC and SOR of the physical mixture is as follows.

Differential Scanning Calorimetry, or DSC, is a thermal analysis technique that looks at how a material's heat capacity (Cp) is changed by temperature. A sample of known mass is heated or cooled and the changes in its heat capacity are tracked as changes in the heat flow. This allows the detection of transitions such as melts, glass transitions, phase changes, and curing.

### Materials and Methods

DSC (2° C./min), onset ° C.
Apparatus: TA Q100 or Equivalent
Sample Preparation: 2.0-5.0 mg of the test sample was weighed and transferred into aluminium hermic pan, lid was closed and sealed with crimper. Hold it in the sample compartment.
Furnace temperature program: 30 to 150° C. at 2° C./min (Pilocarpine-(R)-Lipoate)
   : 30 to 300° C. at 2° C./min (Physical mixture of Pilocarpine HCl & (R)-Lipoic acid).

### pH (1% Solution)

Apparatus: Balance, pH meter & 100 ml volumetric flask
Sample Preparation: 1 g of the test sample was weighed and transferred to 100 ml volumetric flask, 50 ml water was added for volume make up. pH of the sample was checked with pH meter. Specific Optical Rotation, SOR is optical rotation analysis of physical mixture of R-Lipoic acid and piloparpine HCl.

Specific Optic Rotation, [α]D 25 (C=1% in Methanol)
Apparatus: Balance, Polarimeter and 50 mL volumetric flask
Procedure: 0.5 g of the sample was weighed accurately in to 50 ml volumetric flask, dissolved and volume made up with methanol.

Calculation:

$$\begin{matrix}t \\ [ α ] \\ λ\end{matrix} = \frac{100 \times a}{1 \times c}$$

Where,
[α]=Specific rotation
t=temperature
λ=Wavelength
a=observed rotation
l=Length of cell (decimeters)
c=concentration of sample solution (g/100 mL)

Procedure:
1. Taken small quantity of microscopy oil on clean glass slide and added few particles of sample. Mixed well to form homogenous mixture.
2. Cover slip was placed on the top of mixture gently without bubble formation.
3. The prepared slide was placed on the stage of Nikon polarized microscope and photomicrographs were recorded at 4× and 10× accordingly.
4. After completion of the test; prepared slides were discarded.

Interpretation:
1. Crystals of these batches are anisotropic.
2. Crystal morphology was irregular.
3. Particle size is not uniform which may be due to agglomeration.
4. Birefringence was appeared as multi colored spottings at various spaces of a crystal.
5. In presence of cross polarization crystals appeared to shine in the black background confirming the crystal nature of the material.

TABLE 1
DSC data:

| Sr No | Compound name | Result |
|---|---|---|
| 1 | Physical mixture (pilocarpine HCl | 43.83° C. |
| | and R-(+)-Lipoic acid) | |
| 2 | Pilocarpine-R-Lipoate | 72.34° C. |
| 3 | pilocarpine HCl | 198.22° C. |
| 4 | R-(+)-Lipoic acid | 48.43° C. |

TABLE 2
SOR data:

| Sr No | Compound name | Result |
|---|---|---|
| 1 | Physical mixture (pilocarpine HCl | +101.8° |
| | and R-(+)-Lipoic acid) | |
| 2 | Pilocarpine-R-Lipoate | +115.1° |
| 3 | pilocarpine HCl | +89.3° |
| 4 | R-(+)-Lipoic acid | +111.9° |

We claim:

1. A pharmaceutical composition comprising:
a) a therapeutically effective amount of an antimuscarinic or an anticholinergic agent or a pharmaceutically acceptable salt or a stereoisomer thereof; and
b) a therapeutically effective amount of lipoic acid or a pharmaceutically acceptable salt or stereoisomer or prodrug thereof.

2. The pharmaceutical composition as claimed in claim 1, wherein the antimuscarinic or anticholinergic agent is selected from a compound of Formula I: 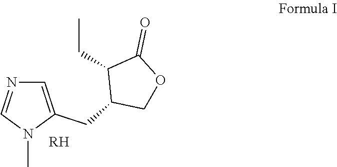
or a pharmaceutically acceptable salt or a stereoisomer thereof, wherein
RH is 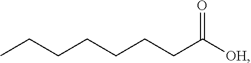
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid;
a compound of Formula II: 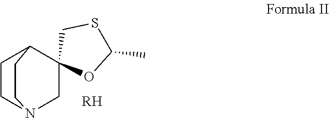
or a pharmaceutically acceptable salt or stereoisomer thereof, wherein,
RH is 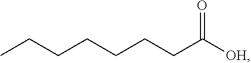
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid; or
a compound of Formula III: 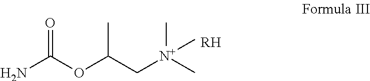
or a pharmaceutically acceptable salt or a stereoisomer thereof; wherein,
RH is 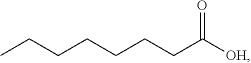
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

3. The pharmaceutical composition as claimed in claim 1, wherein the lipoic acid is a compound of Formula IV: 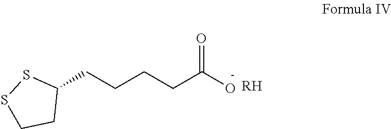
or a pharmaceutically acceptable salt or stereoisomer thereof; wherein,
RH is null, H, sodium, potassium, magnesium, calcium, arginine, glutamate, lysine, glycine, proline, pyridoxine, pyridoxamine, choline, taurine, malic acid, PHMB, polyhexanide or guanidine; or
the lipoic acid prodrug is choline ester prodrug compound of Formula V: 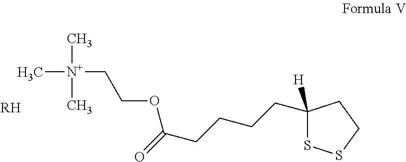 [SMILES: "RH" ; C[N+](C)(C)CCOC(=O)CCCC[C@@H]1CCSS1]
or a pharmaceutically acceptable salt or a stereoisomer thereof; wherein,
RH is H, chloride, iodine, glutamic acid, aspartic acid, lysine, ketorolac, ketoprofen, naproxen, bromine, diclofenac, nepafenac, bromfenac or glycine.

4. The pharmaceutical composition as claimed in claim 1, wherein the antimuscarinic or an anticholinergic agent or pharmaceutically acceptable salt or a stereoisomer thereof, is present in a therapeutically effective dose range of 0.1 mg to 200 mg.

5. The pharmaceutical composition as claimed in claim 1, wherein the lipoic acid or a pharmaceutically acceptable salt or a stereoisomer thereof, is present in a dose of from 10 mg to 2 g.

6. The pharmaceutical composition as claimed in claim 1, wherein the effective dose of the compounds is in the range of about 0.01 mg/kg body weight/day to about 100 mg/kg body weight/day.

7. A physical mixture comprising a therapeutically effective amount of an antimuscarinic or an anticholinergic agent or a pharmaceutically acceptable salt or a stereoisomer thereof; and a therapeutically effective amount of lipoic acid or a pharmaceutically acceptable salt or stereoisomer or prodrug thereof.

8. The physical mixture as claimed in claim 7, wherein the antimuscarinic or anticholinergic agent is selected from a compound of Formula I: 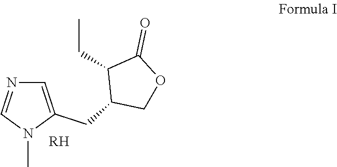
or a pharmaceutically acceptable salt or a stereoisomer thereof, wherein
RH is 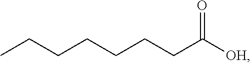
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid;
a compound of Formula II: 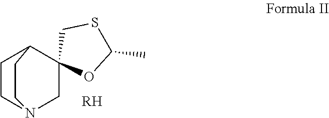
or a pharmaceutically acceptable salt or stereoisomer thereof, wherein,
RH is 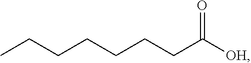
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid; or
a compound of Formula III: 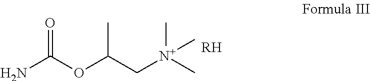
or a pharmaceutically acceptable salt or a stereoisomer thereof; wherein,
RH is 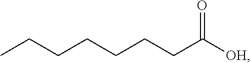
hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

9. The physical mixture as claimed in claim 7, wherein the lipoic acid is a compound of Formula IV: 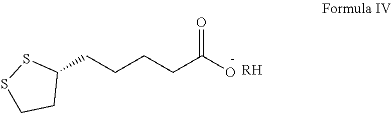
or a pharmaceutically acceptable salt or stereoisomer thereof; wherein,
RH is null, H, sodium, potassium, magnesium, calcium, arginine, glutamate, lysine, glycine, proline, pyridoxine, pyridoxamine, choline, taurine, malic acid, PHMB, polyhexanide or guanidine; or
the lipoic acid prodrug is choline ester prodrug compound of Formula V: 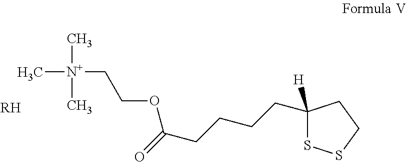 [SMILES: "RH" ; C[N+](C)(C)CCOC(=O)CCCC[C@@H]1CCSS1]
or a pharmaceutically acceptable salt or a stereoisomer thereof; wherein,
RH is H, chloride, iodine, glutamic acid, aspartic acid, lysine, ketorolac, ketoprofen, naproxen, bromine, diclofenac, nepafenac, bromfenac or glycine.

10. The physical mixture of claim 7 comprising a compound of Formula I, and a compound of Formula IV or V.

11. The physical mixture of claim 7 comprising a compound of Formula II, and a compound of Formula IV or V.

12. The physical mixture of claim 7 comprising a compound of Formula III, and a compound of Formula IV or V.

13. The physical mixture of claim 10, wherein the compound of Formula I is pilocarpine HCl and the compound of Formula IV is R-(+)-Lipoic acid.

14. A pharmaceutical composition comprising a compound of Formula I and a compound of Formula IV or a physical mixture thereof.

15. The pharmaceutical composition of claim 15, wherein compound of Formula I is present in a therapeutically effective dose range of 0.01 mg to 200 mg and the compound of Formula IV is present in a therapeutically effective dose range from 5 mg to 4 g.

16. The pharmaceutical composition of claim 14, wherein the compound of Formula I is pilocarpine HCl and the compound of Formula IV is R-(+)-Lipoic acid or the physical mixture thereof.

17. The pharmaceutical composition as claimed in any of claims 1-6 or 14-16 further comprising at least one pharmaceutically acceptable excipient.

18. The composition as claimed in claim 17, wherein said composition is formulated for oral, nasal, dermal, ocular, topical, rectal, vaginal, aerosol or parenteral administration.

19. The composition as claimed in claim 18, wherein said composition is for the treatment of xerostomia, and burning mouth syndrome or a complication thereof.

20. The composition as claimed in claim 18, wherein said composition is for the treatment of the ocular disease or disorder selected from the group consisting of presbyopia, glaucoma and its related conditions.

21. A method of treating xerostomia and complication thereof in a subject of need thereof, the method comprising administering to said subject a therapeutically effective amount of the composition as claimed in any of claims any of claims 1-6 or 14-20.
